(19) United States
(12) Patent Application Publication
(10) Pub. No.: US 2025/0268493 A1
(43) Pub. Date: Aug. 28, 2025

(54) SYSTEM AND METHOD FOR CAMERA-BASED REMOTE BLOOD OXYGEN SATURATION MONITORING

(71) Applicant: The Hong Kong University of Science and Technology, Hong Kong (CN)

(72) Inventors: Kwan Long WONG, Hong Kong (CN); Richard Hau Yue SO, Hong Kong (CN); Jing Wei CHIN, Hong Kong (CN); Tsz Tai CHAN, Hong Kong (CN); Chun Hong CHENG, Hong Kong (CN)

(21) Appl. No.: 19/063,326

(22) Filed: Feb. 26, 2025

Related U.S. Application Data
(60) Provisional application No. 63/557,603, filed on Feb. 26, 2024.

(51) Int. Cl. A61B 5/1455; A61B 5/00; A61B 5/103; G06V 10/25; G06V 10/30; G06V 10/77; G06V 40/10; G06V 40/16; G16H 15/00; G16H 40/67

(52) U.S. Cl. CPC A61B 5/14551; A61B 5/1032; A61B 5/7267; G06V 10/25; G06V 10/30; G06V 10/7715; G06V 40/15; G06V 40/165; G06V 40/171; G16H 15/00; G16H 40/67

(57) ABSTRACT

A system includes a camera, a color image and signal processing system, a physiological activity image processing system, a blood oxygen saturation estimator, and a report output module. The camera captures color image frames of a subject. The color image and signal processing system extracts physiological signals by analyzing light intensity, detecting and resizing facial images, interpolating facial landmarks, creating facial patches, selecting patches based on landmarks, and extracting signals from different color channels. The physiological activity image processing system generates a PAI embedding the subject's physiological information by forming 1D signals, concatenating filtered 1D signals into 2D images for each color channel, and combining the 2D images along a color channel dimension. The blood oxygen saturation estimator, using a deep learning model, receives the PAI to estimate the subject's blood oxygen saturation level. The report output module provides a readable report of the estimation.

### TECHNICAL FIELD

The present invention is related to the field of computer vision that is used to extract physiological-related information from images and then estimate blood oxygen saturation of a subject with the use of a deep learning/machine-learning model.

### BACKGROUND

Human vital signs, such as blood oxygen saturation (SpO₂), heart rate, respiration rate, blood pressure, and body temperature, are standard parameters used to evaluate a person's health status. Specifically, SpO₂ readings indicate whether a person has enough oxygen to operate efficiently. SpO₂ readings are a common metric for trauma management and early detection of diseases like hypoxemia, sleep apnea and heart diseases. Currently, SpO₂ is generally measured non-invasively using pulse oximeters. However, such contact-based devices have usability limitations and are impractical for long-term monitoring. Usage for extended periods can cause discomfort and are unsuitable for those with skin sensitivity. Moreover, using contact-based devices for health monitoring may facilitate the spread of infectious diseases. Therefore, contactless approaches for SpO₂ measurement have emerged as highly desirable.

Over the last decade, several contactless SpO₂ measurement approaches have been proposed. Researchers have used a variety of cameras, from infrared cameras and high-quality monochrome cameras equipped with special filters to off-the-shelf webcams, to estimate SpO₂ by capturing subtle light intensity changes on the face, followed by the analytical Ratio-of-Ratio principle. However, remote deep learning-based SpO₂ measurement is still in its infancy, while deep learning techniques have already achieved state-of-the-art performance for other vital signs such as heart rate.

Accordingly, there is a need for a contactless SpO₂ measurement system that leverages deep learning techniques to enhance accuracy, offering a more comfortable and hygienic solution for continuous health monitoring.

### SUMMARY OF INVENTION

It is an objective of the present invention to provide a system and a method to solve the aforementioned technical problems for extraction of physiological-related information from images.

Briefly, to extract the subject's SpO₂, the system first starts capturing a color video of the subject. Then, computer vision techniques are applied on video frames to locate regions of interest on the face and these regions are being tracked continuously for a while. Next, the images with located regions of interest are fed into a pipeline with image and signal processing algorithms to create a Physiological Activity Image (PAI). This PAI, with embedded physiological information of the subject, is then fed into a deep learning model to predict the SpO₂ in percentage (%) of the subject.

In accordance with a first aspect of the present invention, a system for remote deep learning-based blood oxygen saturation monitoring is provided. The system includes a camera, a color image and signal processing system, a physiological activity image processing system, a blood oxygen saturation estimator, and a report output module. The camera is configured to capture color image frames of a subject. The color image and signal processing system is configured to extract physiological signals according to the color image frames of the subject by: executing light intensity analysis for the color image frames of the subject; identifying a location of a face and facial landmarks within the color image frames to detect facial images; resizing the detected facial images and interpolating the approximated facial landmarks; creating facial patches from the resized facial images; selecting the facial patches based on the interpolated facial landmarks; and setting different color channels and then extracting the physiological signals from the selected facial patches. The physiological activity image processing system is configured to use the extracted physiological signals to generate a PAI embedding the subject's physiological information by: forming 1D (one-dimension) signals from the extracted physiological signals and applying bandpass filtering to each 1D signal; concatenating the filtered 1D signals with the same color channel sequentially to create at a 2D (two-dimension) image for each of the color channels; and concatenating the 2D images of different color channels along a color channel dimension to generate the PAI. The blood oxygen saturation estimator is configured to receive the PAI for inference, resulting in an estimated blood oxygen saturation level of the subject, wherein the blood oxygen saturation estimator has a deep learning model set up for SpO₂ estimation. The report output module is configured to receive an inference result with estimated blood oxygen saturation and provided a readable report regarding the subject's blood oxygen saturation status according to the inference result.

In accordance with a second aspect of the present invention, a method for remote deep learning-based blood oxygen saturation monitoring is provided. The method includes steps as follows: capturing color image frames of a subject using a camera; extracting physiological signals according to the color image frames of the subject using a color image and signal processing system by steps of: (1) executing light intensity analysis for the color image frames of the subject; (2) identifying a location of a face and facial landmarks within the color image frames to detect facial images; (3) resizing the detected facial images and interpolating the approximated facial landmarks; (4) creating facial patches from the resized facial images; (5) selecting the facial patches based on the interpolated facial landmarks; and (6) setting different color channels and then extracting the physiological signals from the selected facial patches. The steps further include: using the extracted physiological signals to generate a PAI embedding the subject's physiological information, using a physiological activity image processing system, by steps of: (1) forming 1D signals from the extracted physiological signals and applying bandpass filtering to each 1D signal; (2) concatenating the filtered 1D signals with the same color channel sequentially to create at a 2D image for each of the color channels; and (3) concatenating the 2D images of different color channels along a color channel dimension to generate the PAI. The steps further include: receiving the PAI for inference by the blood oxygen saturation estimator, resulting in an estimated blood oxygen saturation level of the subject, wherein the blood oxygen saturation estimator has a deep learning model set up for SpO₂ estimation; and receiving an inference result with estimated blood oxygen saturation and providing a readable report regarding the subject's blood oxygen saturation status according to the inference result, using a report output model.

By the configuration, a contactless method for SpO₂ estimation is provided. The solution provided by the present invention enables a contactless method to predict SpO₂ directly by capturing the face of a subject with the use of PAI. The provided contactless method can work with common digital imaging devices such as webcams or smartphone cameras, demonstrating its potential for integration into telehealth services, wellness programs, clinical applications, and even insurance assessments, enhancing accessibility and efficiency across healthcare and related industries.

### BRIEF DESCRIPTION OF DRAWINGS

Embodiments of the invention are described in more details hereinafter with reference to the drawings, in which:

FIG. 1 shows a schematic diagram of an architecture of a remote deep learning-based blood oxygen saturation monitoring system according to some embodiments of the present invention;

FIG. 2 shows a schematic diagram of a process of estimating blood oxygen saturation of a subject according to some embodiments of the present invention;

FIG. 3 shows a schematic diagram of a process for converting raw color frames into physiological signals using a color image and signal processing system according to some embodiments of the present invention;

FIG. 4 illustrates an absorption coefficient of oxygenated hemoglobin and deoxygenated hemoglobin under different wavelengths;

FIG. 5 shows a diagram for forming PAIs from extracted physiological signals using a physiological activity image processing system according to some embodiments of the present invention;

FIG. 6 illustrates a process of forming a physiological activity image from a captured video of a subject according to some embodiments of the present invention; and

FIG. 7 illustrates a process of training a deep learning model for blood oxygen saturation estimation from a captured video of a subject according to some embodiments of the present invention.

### DETAILED DESCRIPTION OF THE INVENTION

In the following description, systems and methods for camera-based remote blood oxygen saturation monitoring and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

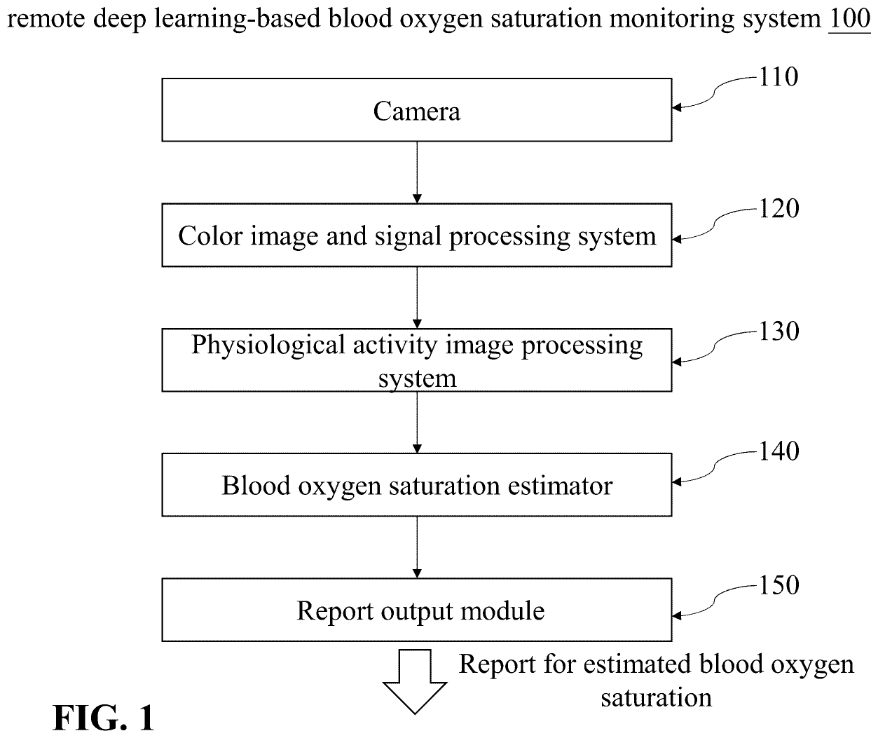

FIG. 1 illustrates a schematic diagram of an architecture of a remote deep learning-based blood oxygen saturation monitoring system 100 according to some embodiments of the present invention. The system 100 includes a camera 110, a color image and signal processing system 120, a physiological activity image processing system 130, a blood oxygen saturation estimator 140, and a report output module 150.

The camera 110 is configured to capture color image frames of a subject to be analyzed. Herein, the term “subject” refers to a human undergoing blood oxygen saturation monitoring. The camera 110 serves as a primary input device for the system 100, providing raw visual data required for subsequent processing steps. In some embodiment, the camera 110 may record one or more color videos with color image frames for the subject.

The color image and signal processing system 120 communicates with the camera 110, in which the color image frames captured by the camera 110 serve as input to the color image and signal processing system 120. The color image and signal processing system 120 is configured to extract physiological signals according to the color image frames of the subject. Specifically, during the processing on the color image frames, the color image and signal processing system 120 can performs several key functions. The color image and signal processing system 120 is configured to: execute light intensity analysis for the color image frames of the subject and then identify the location of the face and facial landmarks; resize detected facial images and interpolate the approximated facial landmarks; create facial patches from the resized facial images; select specific facial patches based on the interpolated facial landmarks; and, extract physiological signals from the selected facial patches, providing essential data for subsequent stages of the process.

The physiological activity image processing system 130 communicates with the color image and signal processing system 120, in which the physiological signals are transferred to the physiological activity image processing system 130 from the color image and signal processing system 120 after extraction. The physiological activity image processing system 130 is configured to use the extracted physiological signals to generate a physiological activity image (PAI) embedding the subject's physiological information. Specifically, during the generation of the PAI, the physiological activity image processing system 130 can performs several key functions. The physiological activity image processing system 130 is configured to: form 1D (one-dimension) signals from the extracted physiological signals and apply bandpass filtering to each 1D signal; concatenate the filtered 1D signals with the same color channel sequentially to create a 2D (two-dimension) image; and concatenate the 2D image of different color channels along the color channel dimension to generate a PAI.

The blood oxygen saturation estimator 140 communicates with the physiological activity image processing system 130 for receiving the PAI. The PAI can be fed into the blood oxygen saturation estimator 140 for inference, resulting in an estimated blood oxygen saturation level of the subject. Specifically, the blood oxygen saturation estimator 140 includes a deep learning model set up for SpO₂ estimation. The deep learning model can be trained for SpO₂ estimation using a SpO₂ training set that incorporates processed PAIs. Once the deep learning model has been trained, the blood oxygen saturation estimator 140 is configured to utilize the trained model to estimate SpO₂ for the subject based on the input PAI from the physiological activity image processing system 130.

The report output module 150 communicates with the blood oxygen saturation estimator 140 for receiving an inference result with estimated blood oxygen saturation. The report output module 150 is configured to compile and present the result in a user-friendly format, providing a readable report regarding the subject's blood oxygen saturation status.

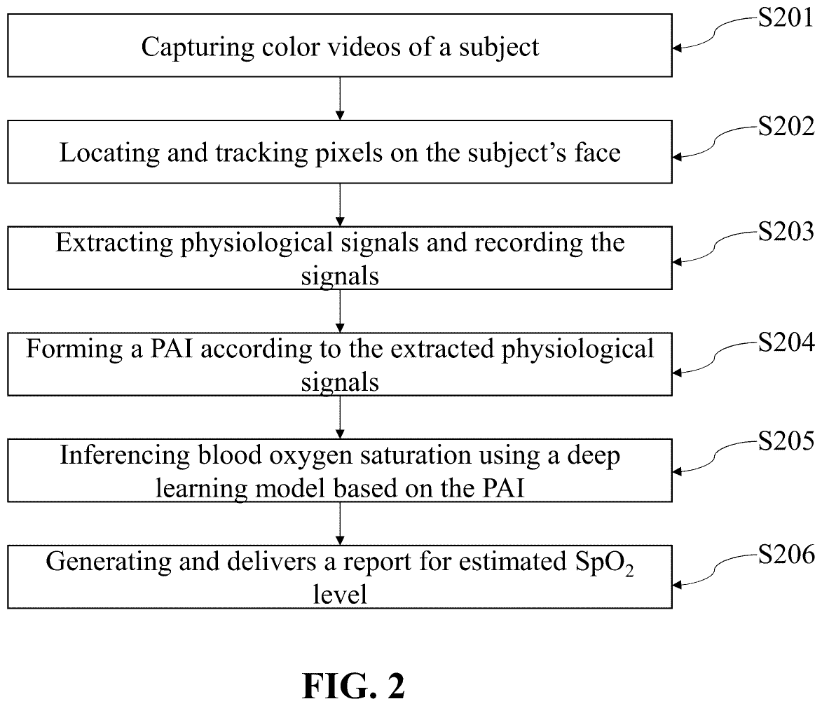

FIG. 2 shows a schematic diagram of a process of estimating blood oxygen saturation of a subject according to some embodiments of the present invention. The process shown in FIG. 2 can be executed using the remote deep learning-based blood oxygen saturation monitoring system 100 as afore-mentioned in FIG. 1. The process includes steps S201, S202, S203, S204, S205, S206 for estimating SpO₂ of a subject.

The process begins with step S201, which involves capturing color videos of a subject. During this step, the camera 110 is activated to record color videos for the subject, obtaining raw color frames that are essential for subsequent stages of physiological signal extraction.

In step S202, the raw color frames captured by the camera 110 are transmitted to the color image and signal processing system 120 for feature extraction and region identification. The color image and signal processing system 120 locates and tracks pixels on the subject's face and identifies regions of interest (ROIs) within the raw color frames.

Once the ROI of the subject's face is determined, the process moves to step S203. The color image and signal processing system 120 extracts physiological signals from the ROI based on the color image frames and records these signals. During this stage, the transformation of raw color frames into physiological signals, executed by the color image and signal processing system 120, helps minimize noise in the frames and provides higher-quality data for physiological signal extraction.

Next, the process advances to step S204. In step S204, the extracted physiological signals are transmitted from the color image and signal processing system 120 to the physiological activity image processing system 130. The physiological activity image processing system 130 processes the physiological signals to form a physiological activity image (PAI), which serves as a single 2D input embedding the subject's physiological information.

After formation/generation of the PAI, the process proceeds to step S205. The PAI is input into the blood oxygen saturation estimator 140, such that SpO₂ can be estimated by feeding the 2D input into the blood oxygen saturation estimator 140 for model prediction. The blood oxygen saturation estimator 140 utilizes the PAI to infer blood oxygen saturation, specifically predicting the subject's SpO₂ level. This stage employs deep learning techniques, and the blood oxygen saturation estimator 140 applies a well-trained model.

Finally, in step S206, the estimated SpO₂ level made by the blood oxygen saturation estimator 140 is output to the report output module 150. The report output module 150 generates and delivers a report, completing the process of estimating the subject's SpO₂.

The following will describe the details of how each component processes the data.

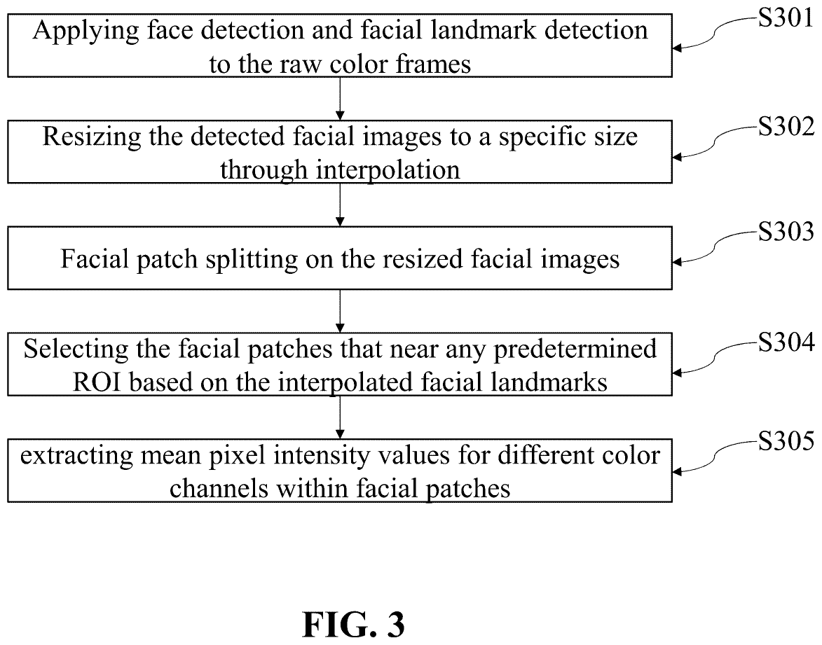

FIG. 3 shows a schematic diagram of a process for converting raw color frames into physiological signals using a color image and signal processing system according to some embodiments of the present invention. The process includes steps S301, S302, S303, S304, S305 for the converting.

Once the color image and signal processing system 120 receives the raw color frames of the subject from the camera 110, the process begins with step S301. In this step, the color image and signal processing system 120 applies face detection and facial landmark detection to the raw color frames, thereby obtaining detected facial images with ROIs of a face representation. This is done since the ROIs on the face representation are used for extracting physiological signals in the next step. Within the face representation, the ROIs are located by computer vision algorithms that approximate the facial landmarks on the face representation of the subject, which are descriptors for the position of face features, such as nose, eyes, mouth, etc.

The color image and signal processing system 120 identifies the location of the face and facial landmarks of the subject using any kind of computer vision technique for face detection and facial landmarks detection. In some embodiments, the color image and signal processing system 120 includes a face detection model (e.g., a pre-trained convolutional neural network (CNN)-based face detection model) for face detection and a facial landmark predictor.

In the face detection and facial landmarks detection process, the raw color frames are first processed using the face detection model, which detects the bounding box of the face representation within the raw color frames. Once the face representation is localized, the facial landmark predictor applies a detection algorithm to identify key facial points, such as areas around the eyes, the tip of the nose, and regions near the mouth, and to determine facial landmarks, defining ROIs on the face representation for subsequent analysis.

The process moves to step S302. The color image and signal processing system 120 processes the detected facial images by resizing them to a specific size through interpolation, thereby obtaining resized facial images. Simultaneously, for consistency, the corresponding estimated facial landmarks are interpolated using the color image and signal processing system 120 to maintain alignment with the resized facial images.

In some embodiments, the color image and signal processing system 120 can resize the detected facial images to any specific size using various interpolation techniques. For example, the color image and signal processing system 120 includes an interpolation model employing bilinear interpolation or bicubic interpolation to achieve smooth scaling while preserving the visual quality of the facial images.

In some embodiments, the color image and signal processing system 120 can interpolate the approximated facial landmarks using any suitable interpolation technique. For example, the interpolation model of the color image and signal processing system 120 further employs linear interpolation or spline-based methods to adjust the spatial positions of the facial landmarks relative to the resized facial images, achieving correspondence between the landmarks and the adjusted facial dimensions.

The process moves to step S303. The color image and signal processing system 120 executes facial patch splitting on the resized facial images. Each resized facial image is divided into multiple facial patches, making all facial patches be the same size (e.g., the same dimensions in pixels). The facial patches are labeled sequentially based on their locations. Furthermore, the facial patches from different color frames that occupy the same location are assigned the same label, regardless of the frame they belong to. For example, if there are two color frames, a first color frame and a second color frame; the facial patches at the same location in the first and second color frames share the same label, indicating they are at the same location.

In some embodiments, the color image and signal processing system 120 creates the facial patches from the detected facial image by dividing the detected facial image into facial patches of any specific size. For example, each of the facial patch is a square block (e.g., 32×32 pixels or 16×16 pixels).

The process moves to step S304. The color image and signal processing system 120 selects the facial patches that near any predetermined ROI based on the interpolated facial landmarks. In this regard, the color image and signal processing system 120 may perform the selection by choosing the facial patches that are near any predetermined ROIs as defined by the interpolated facial landmarks which are created in step S301. The predetermined ROIs may include areas around the eyes, the tip of the nose, or regions near the mouth, as these areas often provide more robust physiological signals. The facial patches corresponding to these regions are selected to enhance the extraction processing.

In some embodiments, for naturally paired facial features, such as the left and right eyes or the left and right cheeks, a pair of facial patches can be selected, with each facial patch individually including one of the paired features. For example, two facial patches can be defined, where one covers the left eye and the other covers the right eye, thereby facilitating symmetry or difference analysis of paired features.

The process moves to the final step S305. For each selected facial patch, the color image and signal processing system 120 extracts a mean pixel intensity value for each color channel (e.g., red, green, blue) within the facial patch and the records these values. Once the mean pixel intensity values for all facial patches under all desired color channels have been extracted, the mean pixel intensity values are packaged as physiological signals by the color image and signal processing system 120, completing the process of extracting physiological signals. In other words, the mean pixel intensity values constitute the physiological signals.

The mean pixel intensity values of the physiological signals collected by the color image and signal processing system 120 in steps S301-S305 serve as essential factors for estimating SpO₂.

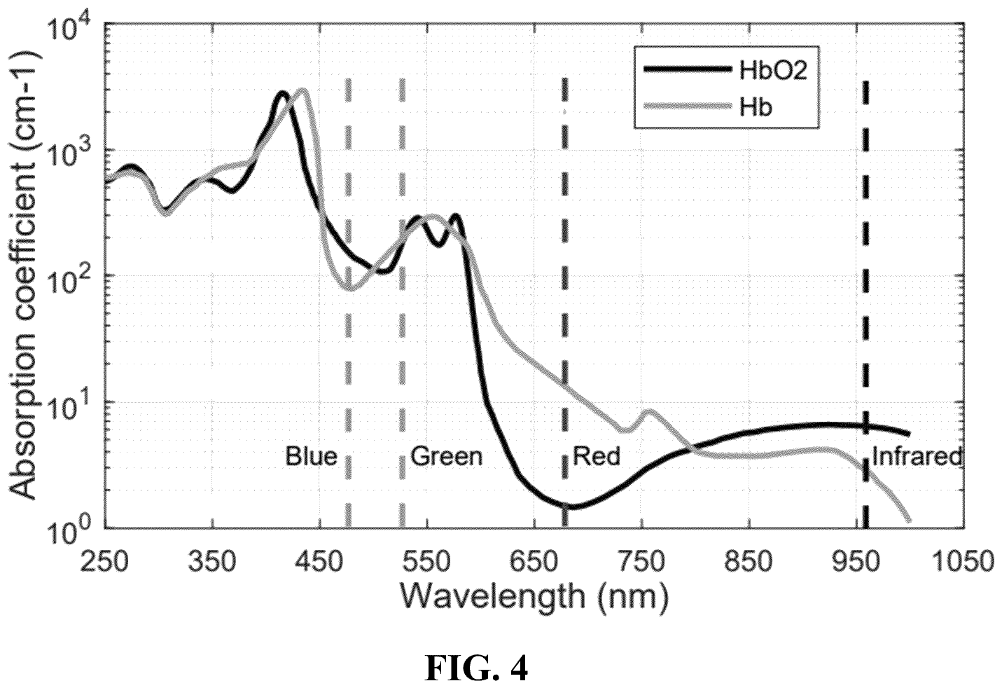

FIG. 4 illustrates an absorption coefficient of oxygenated hemoglobin and deoxygenated hemoglobin under different wavelengths. As referred to FIG. 4, for commercial pulse oximeters, they contain light emitter diodes that generate two different light wavelengths, 660 nm (red) and 960 nm (infrared), to measure the different absorption coefficients of oxygenated hemoglobin (HbO₂) and deoxygenated hemoglobin (Hb). The photodetector inside the pulse oximeter analyzes the light absorption of these two wavelengths and produces an absorption ratio from which the SpO₂, as a %, can be determined. However, since infrared wavelengths cannot be captured in color videos, the infrared component can be replaced by the blue wavelength as the difference between the absorption coefficients of HbO₂ and Hb are very similar at these two wavelengths.

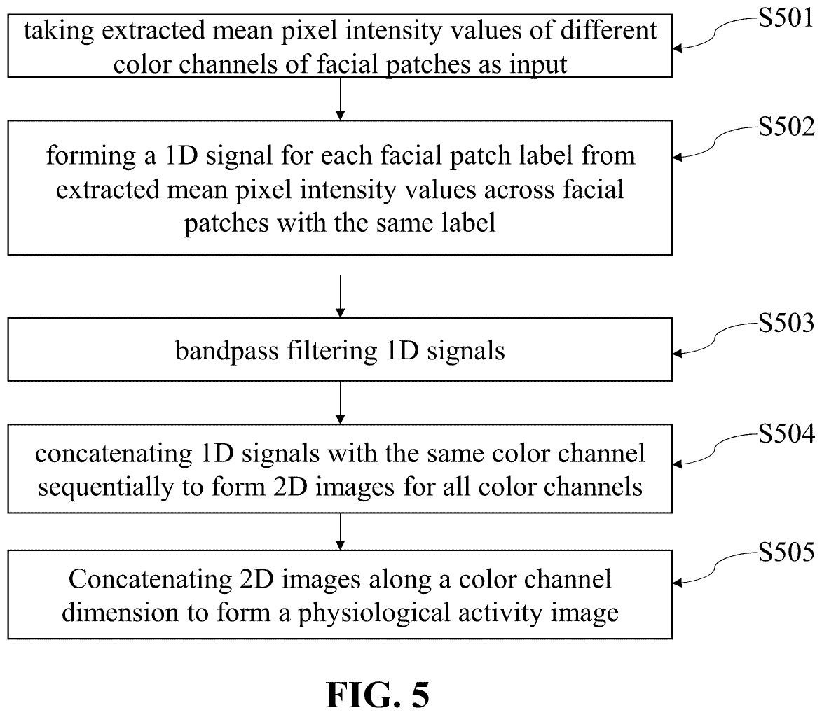

FIG. 5 shows a diagram for forming PAIs from extracted physiological signals using a physiological activity image processing system according to some embodiments of the present invention. The process of forming PAIs include steps S501, S502, S503, S504, and S505.

Once the physiological activity image processing system 130 receives the extracting physiological signals from the color image and signal processing system 120, the process begins with step S501. Based on the physiological signals, the physiological activity image processing system 130 can take the extracted mean pixel intensity values of different color channels of facial patches as input.

The process moves to step S502. For each color channel, the physiological activity image processing system 130 forms a 1D signal for each facial patch label from the extracted mean pixel intensity values across facial patches with the same label. Accordingly, the number of the 1D signals corresponds to the number of the labels for assigning to the facial patches that result from dividing the detected facial image in step S303. For example, if one facial image is divided into N facial patches with N labels to be assigned, the number of the 1D signals is A*N, where A is related to the number of the color channels; A and N are positive integers.

The process moves to step S503. The physiological activity image processing system 130 has a bandpass filter, and the 1D signals are further processed by the bandpass filter to remove noise and retain physiological-related information. In some embodiments, the bandpass filtering performed by the physiological activity image processing system 130 utilizes a bandpass filter with any order, low cutoff frequency, and high cutoff frequency to reduce noise and retain physiological-related information.

The process moves to step S504. The physiological activity image processing system 130 has a concatenator for forming 2D images. Specifically, with respect to each color channel (e.g., red, green, or blue), the 1D signals corresponding to each facial patch label are concatenated sequentially by the concatenator, resulting in a 2D image. As different color channels are applied, multiple 2D images are formed. If the number of color channels is B, the number of 2D images will also be B. In some embodiments, the concatenating the filtered 1D signals with the same color channel sequentially to form a 2D image by the physiological activity image processing system 130 involves arranging these 1D signals in any order with placing them next to each other along a spatial dimension to construct the 2D image.

The process moves to the final step S505. The 2D images of different color channels are further concatenated by the concatenator along a color channel dimension to form a physiological activity image. As such, the concatenation can form a multi-channel physiological activity image, where each channel corresponds to a specific color. In some embodiments, the concatenating the 2D images of different color channels along the color channel dimension to form a PAI by the physiological activity image processing system 130 involves arranging these 2D images in any order along the color channel axis to create the final PAI.

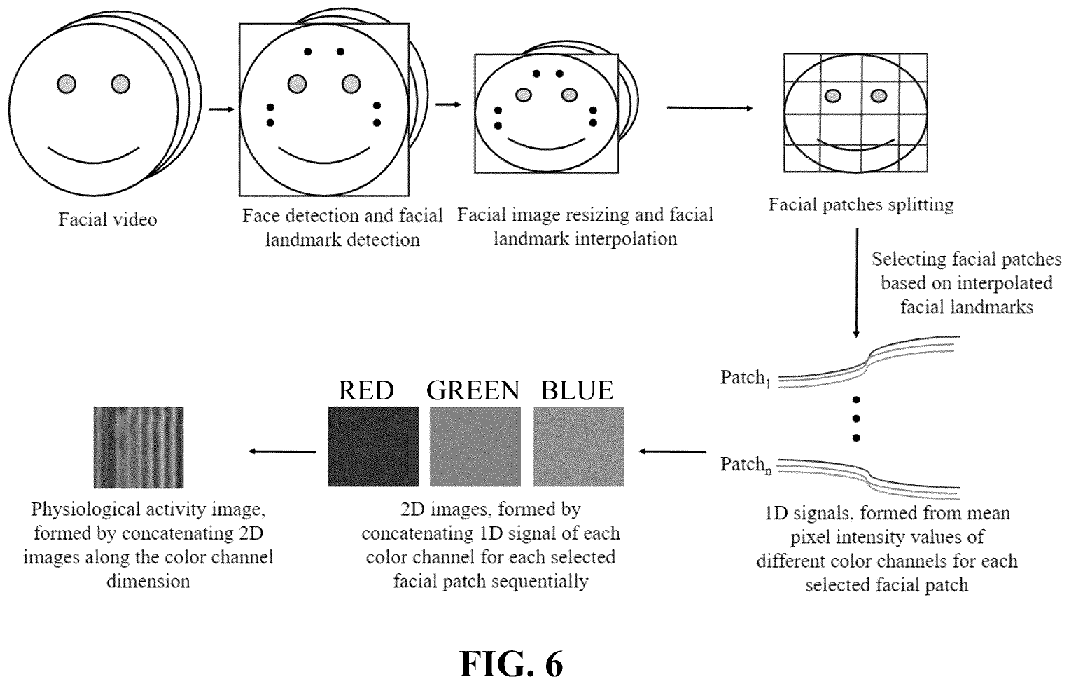

FIG. 6 illustrates a process of forming a physiological activity image from a captured video of a subject according to some embodiments of the present invention. The specific intermediate and final products of steps S301 to S305 and S501 to S505 are shown in FIG. 6.

A facial video serves as input for the process of forming a PAI. Then, the process begins with applying face detection and facial landmark detection on the captured video of the subject. For each video frame, the detected facial image is resized to a specific size by interpolation, and the estimated facial landmarks are also interpolated with respect to the resized facial image. Facial patches splitting is then performed on the resized facial image. Each facial patch is in the same size and it is labelled sequentially based on its location. Moreover, facial patches that are in the same location but from different frames will have the same label.

These facial patches are then further selected if they near any predetermined ROI that located by the interpolated facial landmarks.

From these selected facial patches, 1D signals are formed based on the extracted mean of pixel intensity values of different color channels for each facial patch label. For each color channel, the 1D signal of each facial patch label is concatenated sequentially, resulting a 2D image. Lastly, the PAI is formed by concatenating the 2D image of each color channel along the color channel dimension.

The flowchart in FIG. 6 provides a physical and specific representation of the proposed solution of the present invention.

From the above description, with the provided processes a PAI data point can be obtained for each subject. In the case of a group of subjects, a PAI dataset can be acquired. Additionally, SpO₂ ground truth values of the group of subjects can be obtained through collection methods (not limited to a specific method, such as contact-based). Following this way, the prepared PAI dataset and the corresponding SpO₂ ground truth values dataset can be fed into the blood oxygen saturation estimator 140 for training.

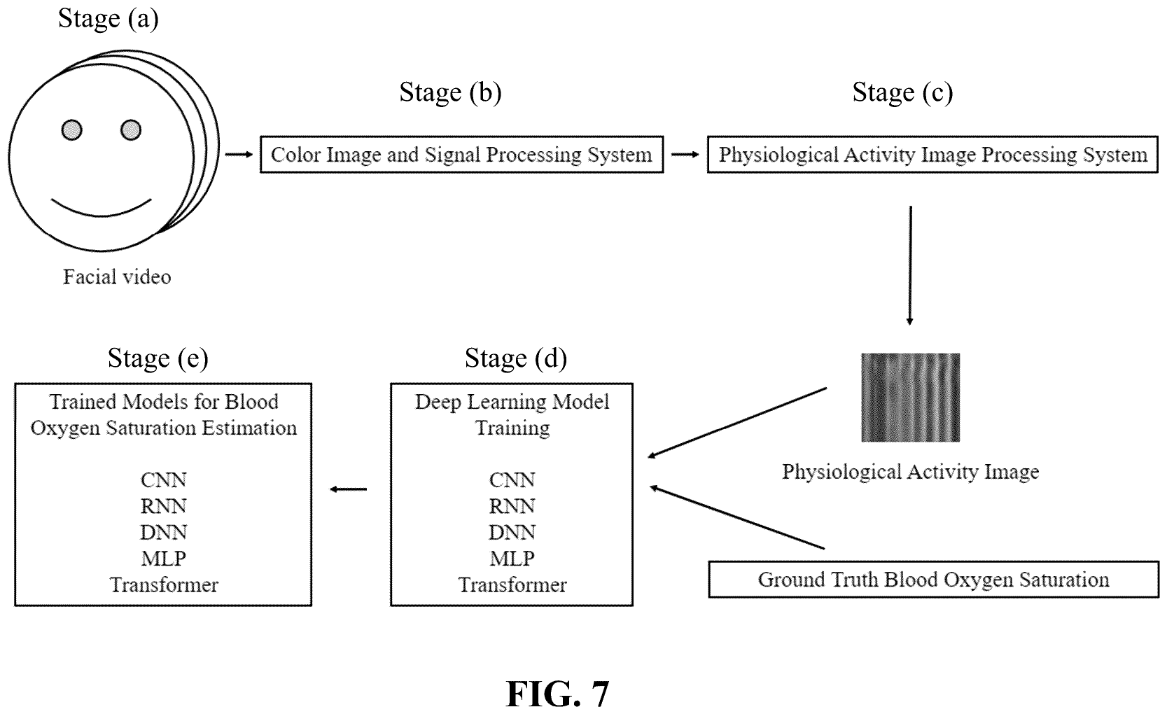

FIG. 7 illustrates a process of training a deep learning model for blood oxygen saturation estimation from a captured video of a subject according to some embodiments of the present invention.

In FIG. 7, before training a deep learning model with PAI for SpO₂ estimation, stages (a), (b), (c) are executed. Stage (a) is obtaining a facial video using the camera 110; stage (b) is extracting physiological signals from facial videos using the with the color image and signal processing system 120; stage (c) is forming one or more PAIs using physiological activity map processing system 130 based on the extracted physiological signals.

After stage (c) and before training, a training set containing SpO₂ ground truth values and processed PAIs which correspond with a group of the same subjects is well-prepared.

During the training for the deep learning model, as stage (d) the processed PAIs and SpO₂ ground truth values are fed into the deep learning model of the blood oxygen saturation estimator 140. The deep learning model can automatically extract features from the PAIs and learn to form a functional relationship (e.g., a loss function) for mapping a PAI to an estimated SpO2. The deep learning model is further configured to utilize the provided SpO₂ ground truth value to guide itself to improve the proposed function. Specifically, the deep learning model calculates the difference between its predicted SpO₂ values and the ground truth using a loss function, such as Mean Squared Error. The calculation to the difference may be performed as an iterative process, allowing the deep learning model to learn and improve its performance over time.

Once the model is trained, the blood oxygen saturation estimator 140 can estimate SpO₂ of the subject using the well-trained deep learning model. After training, as stage (e), by utilizing the functions of the camera 110, the color image and signal processing system 120, and the physiological activity image processing system 130 to process and generate the subject's PAI fed into the blood oxygen saturation estimator 140, the estimated blood oxygen saturation level for any given subject can be obtained using the trained model of the blood oxygen saturation estimator 140.

The functional units and modules of the apparatuses and methods in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes executing in the computing devices, computer processors, or programmable logic devices can readily be prepared by practitioners skilled in the software or electronic art based on the teachings of the present disclosure.

All or portions of the methods in accordance with the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can be included, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A system for remote deep learning-based blood oxygen saturation monitoring, comprising:
a camera configured to capture color image frames of a subject;
a color image and signal processing system configured to extract physiological signals according to the color image frames of the subject by:
executing light intensity analysis for the color image frames of the subject;
identifying a location of a face and facial landmarks within the color image frames to detect facial images;
resizing the detected facial images and interpolating the approximated facial landmarks;
creating facial patches from the resized facial images;
selecting the facial patches based on the interpolated facial landmarks; and
setting different color channels and then extracting the physiological signals from the selected facial patches;
a physiological activity image processing system configured to use the extracted physiological signals to generate a physiological activity image (PAI) embedding the subject's physiological information by:
forming 1D (one-dimension) signals from the extracted physiological signals and applying bandpass filtering to each 1D signal;
concatenating the filtered 1D signals with the same color channel sequentially to create at a 2D (two-dimension) image for each of the color channels; and
concatenating the 2D images of different color channels along a color channel dimension to generate the PAI;
a blood oxygen saturation estimator configured to receive the PAI for inference, resulting in an estimated blood oxygen saturation level of the subject, wherein the blood oxygen saturation estimator has a deep learning model set up for SpO₂ estimation; and
a report output module configured to receive an inference result with estimated blood oxygen saturation and provided a readable report regarding the subject's blood oxygen saturation status according to the inference result.

2. The system according to claim 1, wherein the color image and signal processing system comprises a face detection model and a facial landmark predictor for face detection and facial landmarks detection.

3. The system according to claim 2, wherein the face detection model is configured to process the color image frames for detecting a bounding box of the face representation within the color image frames, and wherein the facial landmark predictor applies a detection algorithm to identify key facial points and to determine the facial landmarks, defining regions of interest (ROIs) on the face representation.

4. The system according to claim 1, wherein the color image and signal processing system is further configured to resize the detected facial images first and then resize the facial landmarks to maintain alignment with the resized facial images for consistency.

5. The system according to claim 1, wherein the color image and signal processing system selects the facial patches near predetermined ROIs as defined by the identifying the location of the face and the facial landmarks, and wherein the predetermined ROIs include areas around eyes, nose, mouth, or combinations thereof.

6. The system according to claim 1, wherein the color image and signal processing system extracts the physiological signals from the selected facial patches via extracting a mean pixel intensity value for each of different color channels within the facial patch.

7. The system according to claim 6, wherein the mean pixel intensity values are packaged as the physiological signals by the color image and signal processing system, completing the extracting the physiological signals.

8. The system according to claim 1, wherein the physiological activity image processing system applies bandpass filtering to each 1D signal via a bandpass filter with any order, low cutoff frequency, or high cutoff frequency to remove noise and retain physiological-related information.

9. The system according to claim 1, wherein the deep learning model of the blood oxygen saturation estimator is trained using a training set comprising processed PAIs for a group of testers and SpO₂ ground truth values of the group of the testers, such that the SpO₂ estimation is presented based on the SpO₂ ground truth values.

10. The system according to claim 9, wherein the deep learning model of the blood oxygen saturation estimator is further configured to extract features from the PAIs in the training set and establish a functional relationship with the SpO₂ ground truth values, thereby mapping the PAI from the physiological activity image processing system to the SpO₂ estimation.

11. A method for remote deep learning-based blood oxygen saturation monitoring, comprising:
capturing color image frames of a subject using a camera;
extracting physiological signals according to the color image frames of the subject using a color image and signal processing system by steps of:
executing light intensity analysis for the color image frames of the subject;
identifying a location of a face and facial landmarks within the color image frames to detect facial images;
resizing the detected facial images and interpolating the approximated facial landmarks;
creating facial patches from the resized facial images;
selecting the facial patches based on the interpolated facial landmarks; and
setting different color channels and then extracting the physiological signals from the selected facial patches;
using the extracted physiological signals to generate a physiological activity image (PAI) embedding the subject's physiological information, using a physiological activity image processing system, by steps of:
forming 1D (one-dimension) signals from the extracted physiological signals and applying bandpass filtering to each 1D signal;
concatenating the filtered 1D signals with the same color channel sequentially to create at a 2D (two-dimension) image for each of the color channels; and
concatenating the 2D images of different color channels along a color channel dimension to generate the PAI;
receiving the PAI for inference by a blood oxygen saturation estimator, resulting in an estimated blood oxygen saturation level of the subject, wherein the blood oxygen saturation estimator has a deep learning model set up for SpO₂ estimation; and
receiving an inference result with estimated blood oxygen saturation and providing a readable report regarding the subject's blood oxygen saturation status according to the inference result, using a report output model.

12. The method according to claim 11, wherein the color image and signal processing system comprises a face detection model and a facial landmark predictor for face detection and facial landmarks detection.

13. The method according to claim 12, further comprising:
processing the color image frames for detecting a bounding box of the face representation within the color image frames using the face detection model; and
executing a detection algorithm by the facial landmark predictor to identify key facial points and to determine the facial landmarks, defining regions of interest (ROIs) on the face representation.

14. The method according to claim 11, further comprising:
resizing the detected facial images first and then resizing the facial landmarks to maintain alignment with the resized facial images for consistency.

15. The method according to claim 11, wherein the color image and signal processing system selects the facial patches near predetermined ROIs as defined by the identifying the location of the face and the facial landmarks, and wherein the predetermined ROIs include areas around eyes, nose, mouth, or combinations thereof.

16. The method according to claim 11, wherein the color image and signal processing system extracts the physiological signals from the selected facial patches via extracting a mean pixel intensity value for each of different color channels within the facial patch.

17. The method according to claim 16, wherein the mean pixel intensity values are packaged as the physiological signals by the color image and signal processing system, completing the extracting the physiological signals.

18. The method according to claim 11, wherein the physiological activity image processing system applies bandpass filtering to each 1D signal via a bandpass filter with any order, low cutoff frequency, or high cutoff frequency to remove noise and retain physiological-related information.

19. The method according to claim 11, further comprising:
training the deep learning model of the blood oxygen saturation estimator using a training set comprising processed PAIs for a group of testers and SpO₂ ground truth values of the group of the testers, such that the SpO₂ estimation is presented based on the SpO₂ ground truth values.

20. The method according to claim 19, further comprising:
extracting features from the PAIs in the training set and establishing a functional relationship with the SpO₂ ground truth values, thereby mapping the PAI from the physiological activity image processing system to the SpO₂ estimation.
